(19) United States
(12) Patent Application Publication
(10) Pub. No.: US 2009/0061470 A1
(43) Pub. Date: Mar. 5, 2009

(54) Use of Human Hepatocytes for Determining Liver Function and Liver Regeneration

(76) Inventor: Martin Sauer, Rostock (DE)

Correspondence Address: JORDAN AND HAMBURG LLP, 122 EAST 42ND STREET, SUITE 4000, NEW YORK, NY, 10168, US

(21) Appl. No.: 12/223,732

(22) PCT Filed: Feb. 7, 2007
(86) PCT No.: PCT/EP2007/001047

§ 371 Date: Aug. 22, 2008

(30) Foreign Application Priority Data
Feb. 7, 2006 (DE) 10 2006 005 526.8

(51) Int. Cl. C12Q 1/44; C12Q 1/02; C12Q 1/32

(52) U.S. Cl. 435/19; 435/29; 435/26

(57) ABSTRACT

The use of human hepatocytes to determine the liver function and the liver regeneration in a test person, in particular a patient, as well as the use of these human hepatocytes as parameters for prognoses. Also, an in-vitro method to determine the liver function of a test person, in particular a patient using human hepatocytes.

The invention on hand relates to the use of human hepatocytes to determine liver function and liver regeneration in a test person, in a patient in particular, as well as the use of these human hepatocytes as a prognostic parameter. Moreover the invention on hand relates to an in-vitro method to determine liver function and liver regeneration in a test person, a patient in particular, using human hepatocytes.

The diseases “Systemic Inflammatory Response Syndrome” (SIRS) with multi-organ failure, severe sepsis und septic shock are related to the development of reversible liver damage which may result in an impairment of liver function. Regular taking of medication, high alcohol consumption and infections may also lead to a severe impairment of the liver function. The diseases described above can in rare cases lead to irreversible liver failure.

A restricted respectively faulty liver function may for example result in a failure of the brain function, such as hepatic encephalopathy. This is caused by the reduced metabolisation of protein breakdown products in the liver. The symptoms of a restricted liver function are for example reduced productivity, weariness, sleepiness and rarely also a feeling of pressure in the epigastrium.

Finally almost 22% of all patients suffering from septic shock develop liver failure (1). By liver failure or liver insufficiency the cessation of liver function culminating in hepatic coma is referred to. Liver failure results in jaundice icterus, hepatic encephalopathy, the hepatorenal syndrome, blood clotting disorders, in hypalbuminemia with ascites, in thrombocytopenia or in endocrinal disorders.

In a study Harbrecht and his colleagues were able to prove that liver failure that had developed during the stay of a patient in hospital resulted in a prolongation of the hospital stay and severely increased mortality (2). The exact reasons for this are not yet clear. Previous explanatory models proceed on the assumption of liver damage resulting from an impairment of the splanchnic perfusion, an endothelium dysfunction or direct damage due to bacterial or other, for example, endogenous toxins (3-6).

Finally it must be noted that in acute liver failure that does not occur within the scope of SIRS or sepsis, systematic and cellular markers of inflammation can be increasingly detected (7).

Sepsis, SIRS with multi-organ failure and liver failure are among the most widely spread causes of death worldwide. In the USA every year approximately 500,000 patients develop sepsis with an average mortality of 21% (8). In Germany about 20,000 patients die of liver disease every year. In the case of a fulminant liver failure the only remaining solution is a liver transplantation.

In all it is extremely important that a restricted respectively faulty liver function in a test person be determined or diagnosed as early as possible to treat or prevent an acute liver failure accordingly at an early stage.

A restricted respectively faulty liver function can currently be diagnosed by measuring various liver-specific and unspecific blood values. Thus by measuring the liver enzymes, Glutamat-Pyruvat-Transaminase (GPT), Glutamat-Oxylacetat-Transaminase (GOT) and Gamma-Glutamyl-Transferase (gamma GT) liver damage can be verified. A failure of the excretoric function and the detoxication function of the liver, for example, results in increased values of bilirubin, gamma GT, ammonia and alcaline phosphatase (AP). In case of reduced synthesis of the liver the concentration of proteins in the plasma such as albumin, for example, and of many coagulation factors may decrease.

The disadvantage of the diagnostic methods currently in use is that findings for many of the blood values mentioned only become clearly pathological with advanced liver failure and are thus unsuited to early diagnosis of liver failure.

To determine liver function further methods are applied that, for example, detect an impairment of the splanchnic perfusion. This can be effected by means of the LiMON-system (PULSION Medical Systems AG, Germany).

Use of the LiMON-system to detect an impairment of the splanchnicus perfusion and of the liver function is currently being clinically tested (3-4). For technical reasons however this method is very difficult to apply to achieve correct measurement results in case of patients suffering from severe sepsis.

In summary it could be said that the methods currently applied only supply relatively unspecific statements concerning the liver function of a patient.

The assignment of the invention is thus to provide a new cytotoxicity test based on human hepatocytes by means of which direct liver damage of a test person and the regeneration of the disease can be ascertained.

According to the invention a new in-vitro method using human hepatocytes respectively a new way of using human hepatocytes is presented to solve the problem. Thus the liver function of a test person, in particular a patient suffering from the Systemic Inflammatory Response Syndrome (SIRS), from sepsis, liver failure or multi-organ failure can be determined at a cellular level.

To date no similar tests at the cellular level to determine the liver function of test persons, in particular of patients with a restricted or faulty liver function, providing the same results that are obtained by the in-vitro method according to the invention are known. In particular no cytotoxicity test based on human hepatocytes, recording the direct damage of the liver of a patient suffering from SIRS with a multi-organ failure, or from severe sepsis or a septic shock and from liver failure on the cellular level, not known.

The in-vitro method according to the invention respectively the use of human hepatocytes to determine the liver function on a cellular level according to the invention has the advantage that here statements about the liver function of a test person, in particular a patient can be obtained much more precisely and much earlier than by the methods currently applied.

It must be pointed out that in the following the term “test person” includes a “healthy test person” as well as a “sick test person” in the sense of a patient.

The in-vitro method according to the invention respectively the use of human hepatocytes to determine the liver function according to the invention are based on the principle that each substance can have toxic effects on human cells subject to its concentration and its combination with other substances. By means of the method respectively its use according to the invention a cytotoxic test is thus provided recording endogenous and exogenous cytotoxicity (for instance from medication).

According to the invention human hepatocytes with plasma from test persons are incubated to determine the liver function. Cell damage of human hepatocytes after incubation with plasma from the test person is considered the parameter for the liver function of a test person. In particular after the incubation of human hepatocytes with plasma from a test person liver function and liver regeneration of a test person can be inferred by means of measuring certain parameters as for example vitality, functionality and activity of the human hepatocytes.

The method to determine the liver function is applied on the basis of human hepatocytes that are preferentially chosen from the human liver cell lines C3A, HLE, HepG2, HuH7 Hep3B, PLC/PRF/5. The respective cell lines were obtained from the American Type Culture Collection (ATCC) and thus comply with general quality standards.

The cytotoxicity test consists of two parts of a testing kit: The first part consists of microtiter plates overlaid with human hepatocytes (24 well-plates). The second part consists of solutions ready for use from agents needed for the test.

To determine the liver function human hepatocytes are incubated with plasma from a test person. The plasma from the test person is practically gained in the form of a cell free solution from the blood with anticoagulants. The coagulation from the blood samples will be supplemented with anticoagulants such as, for example, heparin, heparinoids or substances blocking calcium ions such as citrate, fluoride or oxalate. The recovery of plasma and the anticoagulant concentration and composition needed for its recovery are standardized by an international norm and should thus be known to the practitioner.

The in-vitro method respectively the use of human hepatocytes to determine the liver function according to the invention are based on the principle that certain substances and metabolic metabolites as endogenous and exogenous toxins are accumulating in the plasma of a test person in case of a restricted or faulty liver function. Those substances can then have a toxic effect on the human hepatocytes. By means of determining certain vitality, functionality and activity parameters after incubation of the human hepatocytes with plasma from a test person the liver function of a test person can thus be derived.

So the liver function can be derived after incubation of the human hepatocytes with plasma from a test person by means of measuring the activity of cytochrome P450 isoenzyme 1A2 (CYP1A2) of the human hepatocytes.

The cytochrome P450-super family consists of microsomal haemoproteins that are of central importance for the oxidative, peroxidative and reductive metabolism of a number of endogenous and exogenous substances. The cytochromes are further classified in enzyme families showing a concurrence of the protein sequence of at least 40% and in sub families showing a concurrence of over 55%. Cytochrome P4501A2 (CYP1A2) could to date only be determined in the liver of humans and it constitutes up to 15% of the whole cytochrome concentration there. Its expression depends on various factors like gender, race, genetic polymorphisms and the exposition vis-à-vis inductors, such that there are major inter-individual differences. The task of CYP1A2 is in the main related to xenobiotic metabolism (environmental and food poisons, medication).

The activity of cytochrome P450 1A2, an enzyme involved in the biotransformation of various medications is in fact very low in Hep G2/C3A-cells. It can be increased however by induction with methylcholanthrene to 40 times the initial value (12).

In another embodiment the liver function of the test person can be determined after incubation of the human hepatocytes with his plasma by synthesizing microalbumin and other proteins by means of measuring the activity of the human hepatocytes.

Albumins are highly water-soluble globular proteins. They consist of approximately 580 amino acids and have an average molecular weight of 66,000 Dalton. Albumins account for approximately 60% of all proteins in the blood plasma and they mainly serve to regulate the intravascular oncotic pressure and as transport proteins for water-insoluble substances such as fatty acids, hormones, trace elements and also various medications. Substances with toxic effects reduce the transportability of the albumins by binding with them. Apart from that albumins form an amino acid reserve for the body and contribute to the buffer capacity of the blood. Albumin synthesis occurs only in the liver and represents approximately 10% of the entire synthesis capacity for proteins in the liver. On this account amino acid synthesis is considered an important parameter for the liver function. An adult produces approximately 120-200 mg of albumin/kg body weight daily.

In another embodiment the liver function of the test person can be determined after incubation of the human hepatocytes with plasma from the test person by means of measuring the release of lactate-dehydrogenase by the human hepatocytes.

The lactate-dehydrogenase (LDH) is a cell enzyme that is very important for the intermediary metabolism of the cells as the enzyme is involved in glycolysis. It catalyzes the reaction of pyruvate to lactate. NADH serves as a coenzyme.

Chemical equation:

$$Pyruvate + NADH + {H}^{+} {↔}^{LDH} Lactate + {NAD}^{+}$$

LHD is verifiable in various tissues including in the liver, the heart and skeletal muscles, the erythrocytes, the lymphatic and haematopoietic tissues as well as in the kidney and in malignant tumors.

There are five different isoenzymes with diverse organ allocation. Plasma half-value time of lactate dehydrogenase is 10-120 hours.

The normal value for the LDH activity in the blood serum amounts to 120-240 U/I and results from daily cell turnover.

An increase in this activity is found where the original cells are damaged, whereby enzymes might be released due to permeability failure of the cell membrane or due to lysis of the cells. This can also be the case with increased cell turnover and with regenerative procedures.

In another embodiment the liver function of a test person can be determined after incubation of the human hepatocytes with plasma of the test person by means of measuring the adherence of the vital human hepatocytes.

In another embodiment the liver function of the test person can be determined after incubation of the human hepatocytes with plasma from the test person by means of measuring esterase activity of the vital human hepatocytes.

In another embodiment the liver function of the test person can be determined after incubation of the human hepatocytes with plasma from the test person by means of measuring the number of dead and living human hepatocytes.

In another embodiment the liver function of the test person can be determined after incubation of the human hepatocytes with plasma from the test person by means of measuring the activity of the mitochondrial dehydrogenases of the vital human hepatocytes.

The method respectively the use of human hepatocytes according to the invention is suited to determine the liver function of test persons suffering from the Systemic Inflammatory Response Syndrome (SIRS), from sepsis, from a septic shock, from liver failure or from multi-organ failure.

Furthermore the method respectively the use of human hepatocytes is suited for early diagnosis of a liver failure, to check therapies during the course of the therapy and during regeneration of the liver function.

In addition the method respectively the use of human hepatocytes is suited as parameters for prognosis, by means of which it is possible to prepare a test person for a liver transplantation already at an early stage, in case he for example receives a bad prognosis concerning his liver disease. For test persons with a liver transplantation the method respectively the use according to the invention contributes toward making more accurate statements about the operational capability of the graft after transplantation.

### PREFERRED EMBODIMENTS OF THE INVENTION ARE REFERRED TO BELOW

The method to determine the liver function according to the invention is preferentially carried out on the basis of Hep G2/C3A-cells.

The Hep G2/C3A-cell line (ATCC#CRL-10741) is a sub clone of the widely spread human hepatoblastoma-cell line HepG2.

HepG2/C3A-cells are well distinguished. They possess many phenol and genotypic properties of the normal liver cells and do not carry viral sequences. During the period of growth before confluence the Hep G2&C3A-cells behave similar to the regenerating liver tissue. They have a short duplication time of only 24 hours and secrete a number of fetal proteins including a fetoprotein and adolase A and C and pyruvate kinase K.

As soon as the cells have reached the state of confluence intense inhibition of exposure occurs. Cell division frequency decreases drastically, such that duplication time increases to more than 200 hours and an equilibrium is established. HEp G2/C3A/cells depicting an adult phenotype now prevail. This also becomes clear in protein production: The development of fetal proteins decreases. Instead there is an increased synthesis of albumin, adolase B and pyruvate kinase L.

Apart from promoting the development of various plasma proteins such as, for example, albumin, haptoglobin, coeruloplasmine, α₂-macroglobulin, transferrine, plasminogen, fibrinogen, Hep G2/C3A cells can also effect urea synthesis from lactate and ammonium chloride.

In addition Hep G2/C3A-cells can be cultivated in a glucose-free medium. This shows that the cells mentioned are capable of gluconeogenesis, which is one of the most important liver-specific metabolic functions.

The activity of cytochrome P450 1A2, an enzyme contributing to the biotransformation of various medications is in fact low with Hep G2/C3A-cells. By means of induction with methylcholanthren it can however be increased to 40 times the initial activity (12).

In a test series on the mechanism of tumor invasivity and metastasis various human liver cell lines such as the cell lines C3A, HLE, HepG2, HuH7, Hep3B, PLC/PRF/5 were tested. Here the Hep G2/C3A-cells showed lowest invasivity.

Due to the extensive liver-specific differentiation of the Hep G2/C3A-cell line, the cells were already tested as components of an artificial liver support system to treat liver failure (10).

The HepG2/C3A-cell line is the topic of the U.S. Pat. No. 5,290,684, the disclosure of which is recorded here as reference.

### Cultivating HepG2/C3A-Cells:

HepG2/C3A-cells are cultivated in a drying chamber at 37° C. and with 5% CO₂-gassing, tissue culture bottles with 4 to 20 ml nutrient solution (medium). This consists of Dulbecco's Modified Eagle Medium (Life Technologies Ltd.) with 10% of fetal calf serum

(═FCS, PAA), 1% of glutamine solution (200 mM, Serva) and 1% of antibiotics solution

(Penicillin G (10,000 IE/ml)/Streptomycin (10 mg/ml)—Jenapharm ratiopharm GmbH). For cryopreservation the Hep G2/C3A/cells are stored in liquid nitrogen at 196° C. in tubes containing 1×10⁷ cells in 2 ml medium with 10% of DMSO (dimethyle sulfoxide). A working cell bank was developed. For revitalization the cells are transferred into above mentioned cell medium and after cell counting by means of trypane blue coloring in the Neubauer counting chamber (see below) the cells were seeded in a concentration of 5×10⁵ cells/ml. The first change in medium occurs after 24 hours, after that three times a week. Revitalization time is 14 days. After that the cells are split every 7/10 days and are seeded anew in a concentration of 5×10⁵ cells/ml. The maximum splitting number (passages) of 10 is not exceeded.

### Determination of Parameters

1. Determination of Activity of Cytochrome P450 Isoenzyme 1A2 (Etoxyresorfine-Test Determination of Activity of the Ethoxyresorufine-O-Deethylasis, 11-12)

The activity of cytochrome P450 1A2 can be determined by means of O-deethylization of 7-ethoxyresorufine to resorufine.

For this purpose Hep G2/C3A/cells are seeded in 24 well cell culture plates in a concentration of 250,000 cells/well for three days and 0.5 ml of medium is added. During this period the medium is changed daily. To increase the CYP1A2-activity of the cells induction is effected with 3-methylcholanthren (Sigma) in a concentration of 5.36 g/l (20 μM). From day four to day six the cells are incubated with plasma of the test person (0.5 ml).

The activity is measured on day six after the cells were sown. First the cell culture medium/plasma of the test person is sucked off. Then 0.5 ml of medium with ethoxyresorufine (8 μM) and dicumarol (10 μM) is added to the cells and they must be incubated for one hour at 37° C. in the drying chamber. Thereby a part of the ethoxyresorufine is changed to resorufine by the HepG2/C3A cells. Dicumarol prevents further metabolization of the resorufine.

After that 2×75 μl are taken from each well for duplication and are decanted in a black 96-well-plate. Then 15 μl of a solution of β-glucuronidasis (15 Fishman units) and of arylsulfatasis (120 Roy units) are added to split possibly developed resorufine complexes.

Subsequently a new incubation is effected for three hours at 37° C. by means of shaking.

After that 200 μl of ethanol (99.9%) are pipetted into each well. After another 5 to 10 minutes the resorufine concentration of the samples can be measured by the fluorescence reader

(Fluoroskan Ascent Lab systems) at an excitation of 530 nm and an emission of 584 nm.

In order to convert the fluorescences into resorufine quantities during each test a resorufine standard curve is developed (blank-10 pmol-20 pmol-40 pmol-80 pmol). For each test a clean control of the medium with and without cells and each sample is duplicated.

### Record of the Time Optimization of the Etoxyresorfine Test (Experimental State):

From day eight to day fourteen of cell revitalization 3 methylcholanthren in a concentration of 2.68 g/l (10 μM) is added to the medium. In addition a higher FCS concentration of 15% is applied during the entire revitalization period (14 days). After that the incubation time of the cells with 3 methylcholanthren in a concentration of 5.36 g/l (20 μM) is one day. Incubation time with the plasma of the test person is then only one day, so that a result is available, 48 hours after the start of test.

2. Synthesis Capacity, for Example Micro-Albumin and Other Protein Synthesis of the Hep G2/C3A Cells (13).

The HepG2/C3A-cells are seeded in 24 well cell culture plates in a concentration of 250,000 cells/well for three days and 0.5 ml plasma from test persons+0.25 ml medium is added. The cells are then rinsed once with medium and after that are incubated with fresh medium (1 ml) for three days. After that micro albumin is determined nephelometrically from 0.2 ml cell culture medium and duplicated (+medium control). Each test batch with plasma from test persons is also duplicated.

### Record of the Optimization of the Protein Synthesis Test (Experimental Stage):

By continuously shaking the cell culture bottles using an industrial shaker the cell medium respectively the cell plasma contact is optimized. Consequently the incubation periods with plasma from the test persons as well as with the medium can be shortened to one day, so that a result is available 48 hours after the start of test.

By adding ornithine alpha-cetoglutarates (=OKG, 0.03 mM, 13a) to the medium the synthesis capacity of the Hep G2/C3A-cells is increased. Consequently the test period can also be shortened to 48 hours and the cells release measurable quantities of fibrinogen and transferrine (plasma proteins) that can be measured and duplicated in 200 μl cell culture medium.

3. Determination of the Release of Lactate-Dehydrogenasis (LDH) by the HepG2/C3A Cells (Vitality Test, 14):

The HepG2/C3A-cells are seeded in 24 well cell culture plates in a concentration of 250,000 cells/well for three days and 0.5 ml plasma from test persons+0.25 ml medium is added. The cells are then rinsed once with medium and after that are incubated with fresh medium (1 ml) for three days. After that duplication using 0.2 ml cell culture medium LDH takes place. Each test batch with plasma from test persons is also duplicated. Medium control is also performed.

LDH determination was effected according to the optimized standard method of the Deutsche Gesellschaft für Klinische Chemie (DGKC). Thereby pyruvate is used as a substrate and the NADH decrease is determined photometrically.

### Record of the Time Optimization of the LDH Vitality Test (Experimental Stage):

By continuously shaking the cell culture bottles using an industrial shaker the cell medium respectively the cell plasma contact is optimized. Consequently the incubation periods with the plasma from the test persons as well as with the medium can be shortened to one day, so that a result is available 48 hours after start of test.

### Further Vitality and Proliferation Tests:

The HepG2/C3A-cells are seeded in 24 well cell culture plates in a concentration of 250,000 cells/well for three days and 0.5 ml plasma from test persons+0.25 ml medium is added. The cells are then rinsed once with medium and after that are incubated with fresh medium (1 ml) for three days. Medium control is also performed. After that the following tests are performed with the cells:

4. Adherence Test

First of all the adherence of the HepG2/C3A cells is recorded semi-quantitatively by optical microscope. As the HepG2/C3A cell line is an adherent cell line a large portion of adherent cells in the well speaks for good vitality. The recording of adherence is duplicated.

5. Live/Dead Test

During the live/dead test (molecular probes, 15) 2 colorants (calcein AM and ethidium homodimer) are added to the cells. Calcein-aceton-methylester (Calcein Green AM) is capable of entering the vital cell thanks to membrane permeability and is split intracellularly by esterases within the cell (hydroxylization). The resulting calcein together with calcium ions produces chelates that are unable to leave the cell. The calcein chelates emit light with a wave length of 530 nm at a stimulation wave length of 485 nm. Then a green fluorescence is emitted that can be captured and photographed by a fluorescence microscope. Dead cells have no esterase activity, so very specifically only viable cells show a greenish fluorescence.

For dead cells however the non membrane permeable cell colorant ethidiumbromidhomodimer is suited. This colorant can not pass the intact cell membranes, i.e. can not diffuse through the viable cell, so that this colorant is not absorbed by the viable cells. In dead cells the colorant develops an erythroid fluorescence complex with nucleic acids. (stimulation wave length: 514 nm, emitted wave length: 642 nm)

At the start of the test the medium is carefully sucked off. Then 0.5 ml of live/dead reagent (calcein AM: 2 μM, ethidium homodimer 4 μM) is added. After that the cells are incubated for 30 minutes in a drying chamber at 37° C. under standard conditions. Subsequently the cells are photographed with monochromatic light and the test is evaluated quantitatively. For that purpose a green measurement as well as a red measurement with the above mentioned wave lengths is performed. All measurements are duplicated and a mean value is then determined.

6. XTT-Test

By means of the XTT-test (Roche, 16-17) the transformation of the tetrazolium salt and the resulting change of colour that again results in a change of the extinction value is measured. Thereby the orange colored formazane is developed from the yellow colored tetrazolium salt XTT by means of metabolically active cells. By means of mitochondrial dehydrogenases the transformation of the yellow tetrazolium salt is effected. The transformation of the tetrazolium salt is directly proportional to the activity state of the cells.

At the start of the test the medium is carefully sucked off. Then 0.5 ml of fresh medium and 0.25 ml XTT reagent in a concentration of 1 mg/ml are added. After that the cells are incubated in a drying chamber at 37° C. under standard conditions. The extinctions are measured after 2, 4 and 24 hours (Anthos Reader) and all duplicated (wave length: 450 nm, reference wave length: 690 nm). For this purpose 2×100 μl are taken from each well for duplication and transferred to a 96-well plate.

7. Trypan Blue Vitality Test

After trypsination the cells are coloured with trypan blue and then the number of dead (blue shining)/living (not coloured) cells is recorded.

At the start of the test the medium is carefully sucked off the well and filled in 15 ml blue caps. The possibly existing non-adherent cells are centrifuged off. The centrifuged off medium is discarded. The adherent cells in the well are soaked in 0.5 ml of a trypsine solution (1.25 mg/ml) and 1.5 ml of medium in order to separate the cells. After that they are incubated at 37° C. by means of shaking for 15 minutes. The separated cells are then filled into the prepared blue cap together with the medium in which the possibly non-adherent cells are. The cells are resuspended and then the number of the cells is determined in the Neubauer counting chamber with trypane blue. For this purpose 25 μl of the cell suspension is mixed with 25 μl of trypane blue and then placed in the counting chamber. Two large squares are counted and the result is multiplied by 10,000. This is how the number of living and dead cells is determined. All determinations are duplicated.

### Record on the Time Optimization of the Trypane Blue Vitality Test (Experimental Stage):

By continuously shaking the cell culture bottles using an industrial shaker the cell medium respectively the cell plasma contact is optimized. Consequently the incubation periods with the plasma from a test person as well as with the medium can be shortened to one day, so that a result is provided 48 hours after the start of the test.

### Examples

The cytotoxity test according to the invention was tested on patients suffering from liver failure in order to prove the effectiveness of the liver support method MARS (Molecular Adsorbents Recirculation System, 18) in lowering the number of endogenous toxins which result in further liver damage. For this purpose the plasma samples of the patients were incubated before and after a MARS therapy. With the help of the cytotoxity test obvious differences between the samples of the patients before and after a MARS therapy and in comparison with samples from healthy test persons could be established, in terms of a better function, better synthesis capability and better vitality of the HepG2/C3A cells incubated with plasma from the patients after MARS therapy.

The cytotoxity test according to the invention was applied within the scope of a sepsis therapy study (EISS-I-test) with the extracorporeal plasma perfusion model EISS (Extracorporeal Immune Support System, 19) based on granulocytes from a donor. By means of this test plasma samples from patients suffering from a septic shock were determined during therapy

(four removal times) and in comparison plasma samples from healthy test persons were tested. Obvious differences between the plasma samples of patients and healthy test persons in terms of restricted vitality, synthesis capacity and function of the HepG2/C3A cells of the patients could be determined.

Striking differences were also found in the results with patients between surviving and non surviving HepG2/C3A cells during testing time, so that the cytoxity test developed also has significance for the monitoring of therapy and as a parameter for prognosis.

Currently a large clinical study is being performed, in which the test is being applied to 50 patients suffering from severe sepsis or from a septic shock in comparison with a non-septic community (50 patients). Prior liver disease must be excluded in all patients.

Furthermore the cytoxity test developed is currently being validated within the scope of a study with 15 patients, who must have liver transplantations due to liver failure. The test is intended to help make more accurate statements about the function of the graft in future.

### LITERATURE

(1) Bakker J, Grover R, McLuckie A, Holzapfel L, Andersson J, Lodato R, Watson D, Grossman S, Donaldson J, Takala J; Glaxo Wellcome International Septic Shock Study Group: Administration of the nitric oxide synthase inhibitor NG-methyl-L-arginine hydrochloride (546C88) by intravenous infusion for up to 72 hours can promote the resolution of shock in patients with severe sepsis: results of a randomized, double-blind, placebo-controlled multicenter study (study no. 144-002). Crit Care Med 32 (1):1-12, 2004
(2) Harbrecht B G, Zenati M S, Doyle H R, McMichael J, Townsend R N, Clancy K D, Peitzman A B: Hepatic dysfunction increases length of stay and risk of death after injury. J Trauma 53 (3): 517-23, 2002
(3) Kimura S, et al: Indocyanine green elimination rate detects hepatocellular dysfunction early in septic shock and correlates with survival. Crit Care Med 29: 1159-63, 2001
(4) Sakka S, et al: Prognostic Value of the Indocyanine Green Plasma Disappearance Rate in Critically Ill Patients. Chest 122: 1715-20, 2002
(5) Kobayashi M, Tsujitani S, Kurisu Y, Kaibara N: BcI-2 and Bax expression for hepatocellular apoptosis in a murine endotoxin shock model. Hepatogastroenterology 49 (48): 1602-6, 2002
(6) Nöldge-Schomburg G F E, Priebe H J, Armbruster K, Pannen B, Haberstroh J, Geiger K: Different effects of early endotoxaemia on hepatic and small intestinal oxygenation in pigs. Intensive Care Med 22: 795-804, 1996
(7) Rolando N, Wade J, Davalos M, Wendon J, Philpott-Howard J, Williams R: The Systemic Inflammatory Response Syndrome in Acute Liver Failure. Hepatology 32 (4): 734-9, 2000
(8) Angus D C, Linde-Zwirble W T, Lidicker J, Clermont G, Carcillo J, Pinsky M R: Epidemiology of severe sepsis in the United States: Analysis of incidence, outcome and associated costs of care. Crit Care Med 29 (7): 1303-9, 2001
(9) Kelly J H, Darlington G J: Modulation of the Liver Specific Phenotype in the Human Hepatoblastoma Line HEP G2. In Vitro Cellular and Developmental Biology 25 (2): 217-22, 1989
(10) Ellis A J, Hughes R D, Wendon J A, et al: Pilot-controlled trial of the extracorporeal liver assist device in acute liver failure. Hepatology 24: 1446-51, 1996
(11) Kelly J H, Sussman N L: A Fluorescent Cell-Based Assay for Cytochrome P-450 Isozyme 1A2 Induction and Inhibition. Journal of Biomolecular Screening 5(4): 249-253, 2000
(12) Donato M T, Gómez-Lechón M J, Castell J V: A Microassay for Measuring Cytochrome P450IA1 and P450IIB1 Activities in Intact Human and Rat Hepatocytes Cultured on 96-Well Plates. Analytical Biochemistry 213: 29-33, 1993
(13) Krasteva N, Groth T H, Fey-Lambrecht F, Altankow G: The role of surface wettability on hepatocyte adhesive interactions and function. J Biomater 12 (6): 613-27, 2001
(13a) Lescoat G, Desvergne B, Loreal O, Pasdeloup N, Deugnier Y, Bourel M, Brissot P: Modulation of Albumin Secretion by Ornithine Alpha-Ketoglutarate in Adult Rat Hepatocyte Culture and a Human Hepatoma Cell Line (HepG2). Ann Nutr Metab 33: 252-60, 1989
(14) Modrianski M, Ulrichova J, Bachleda P, Anzenbacher P, Anzenbacherova E, Walterova D, Simanek V: Human Hepatocyte—A Model for Toxicological Studies. Functional and Biochemical Characterization. Gen Phydiol Biophys 19: 223-35, 2000
(15) Dehn P F, White C M, Conners D E, Shipkey G, Cumbo T A: Characterization of the human hepatocellular carcinoma (hepg2) cell line as an in vitro model for cadmium toxicity studies. In Vitro Cell Dev Biol Anim 40 (5-6): 172-82, 2004
(16) Scudiero D A, Shoemarker R H, Paull K D, Monks A, Tierney S, Nofziger T H, Currens M J, Seniff D, Boyd M R: Evaluation of a soluble tetrazolium/formazan assay for cell growth and drug sensitivity in culture using human and other tumor cell lines. Cancer Res 48 (17): 4827-33, 1988
(17) Gressner A M, Polzar B, Lahme B, Mannherz H: Induction of rat liver parenchymal cell apoptosis by hepatic myofibroblasts via transforming growth factor β. Hepatology 23 (3): 571-81, 2003
(18) Sen S, Mookerjee R P, Davies N A, Williams R, Jalan R: Review article: the molecular adsorbents recirculating system (MARS) in liver failure. Aliment Pharmacol Ther 16(5): 32-8, 2002
(19) Mitzner S R, Freytag J, Sauer M, Kleinfeldt T, Altrichter J, Klöhr S, Koball S, Stange J, Ringel B, Nebe B, Schmidt H, Podbielski A, Noeldge-Schomburg G, Schmidt R: Use of human preconditioned phagocytes for extracorporeal immune support: introduction of a concept. Ther Apher 5 (5): 423-32, 2001

1-14. (canceled)

15. A cellular-level test method for determining the liver function of a human test subject comprising the steps of:
a) providing serum plasma from the human test subject,
b) combining the serum plasma from the human test subject with human hepatocytes,
c) incubating the combination of serum plasma from the human test subject and human hepatocytes for an incubating time, and
d) determining at least one cell parameter of the incubated human hepatocytes, the cell parameter being selected from the vitality, functionality, and activity of the incubated human hepatocytes.

16. The cellular-level test method of claim 15 wherein the human hepatocytes are selected from the group consisting of human liver cell lines C3A, HLE, HepG2, HuH7 Hep3B, and PLC/PRF/5, deposited with the American Type Culture Collection.

17. The method of claim 16 wherein the HepG2 human hepatocyte is Hep/C3A.

18. The method of claim 17 wherein the C3A, HLE, HepG2, HuH7 Hep3B, PLC/PRF/5, deposited with the American Type Culture Collection.

19. The cellular-level test method of claim 15 wherein the determining of at least one cell parameter of the incubated human hepatocytes comprises measuring the cytochrome P450 isoenzyme 1A2 activity of the incubated hepatocytes.

20. The cellular-level test method of claim 15 wherein the determining of at least one cell parameter of the incubated human hepatocytes comprises determining the albumin-synthesis activity of the incubated human hepatocytes.

21. The cellular-level test method of claim 15 wherein the determining of at least one cell parameter of the incubated human hepatocytes comprises determining cell vitality by measuring the release of lactate dehydrogenases by the incubated human hepatocytes.

22. The cellular-level test method of claim 15 wherein the determining of at least one cell parameter of the incubated human hepatocytes comprises measuring the mitochondrial dehydrogenases of the incubated human hepatocytes.

23. The cellular-level test method of claim 15 wherein the determining of at least one cell parameter of the incubated human hepatocytes comprises assessing the population of vital, metabolically active cells in the incubated human hepatocytes.

24. The cellular-level test method of claim 23 wherein the population of vital, metabolically active cells in the incubated human hepatocytes is assessed by a method selected from the adherence test, the calcein green AM live/dead test, the XTT-test, and the trypan blue test.

25. A kit for determining the liver function of a human test subject comprising:
a multi-well microtiter plate, overlaid with a plurality of human hepatocytes of one or more human hepatocyte cell lines, and
one or more containers charged with solutions of agents needed for the test, from which container the solutions of agents can be dispensed.

26. The kit of claim 25 wherein the one or more human hepatocyte cell lines are selected from the group consisting of C3A, HLE, HepG2, HuH7 Hep3B, and PLC/PRF/5, deposited with the American Type Culture Collection.

27. The kit of claim 26 wherein the human hepatocyte cell line is the G2C3A cell line know as ATCC CRL-10741.
